(19) United States
(12) Patent Application Publication
(10) Pub. No.: US 2025/0064691 A1
(43) Pub. Date: Feb. 27, 2025

(54) COSMETIC COMPOSITION

(71) Applicant: L’ORÉAL, Paris (FR)

(72) Inventors: Tsang-Min HUANG, Scotch Plains, NJ (US); Jody EBANKS, Clark, NJ (US); Komal LADD, Kenilworth, NJ (US)

(73) Assignee: L’ORÉAL, Paris (FR)

(21) Appl. No.: 18/237,093

(22) Filed: Aug. 23, 2023

(51) Int. Cl. A61K 8/06; A61K 8/34; A61K 8/37; A61K 8/81; A61Q 1/02; A61Q 1/04

(52) U.S. Cl. CPC A61K 8/06; A61K 8/345; A61K 8/37; A61K 8/8105; A61Q 1/02; A61Q 1/04

(57) ABSTRACT

A cosmetic composition may be provided. The composition may include C10-C40 alkyl acrylate polymer and an allylic polymer. The composition may include an emulsifying agent or stabilizer. The composition may be an emulsion. The composition may include a polar solvent, such as a polyol. The polyol may include glycerin, and may include a glycerin ether derivative. The composition may include one or more fatty substances, a polymeric elastomer, a dispersing agent, a hydrocarbon-based resin, a polyolefin, a colorant, a preservative, a skin care agent, an antioxidant, or any combination thereof.

### TECHNICAL FIELD

The present disclosure is drawn to the field of cosmetic compositions, and specifically, to stable, low-tack cosmetic compositions with skin care ingredients that also include both C12-22 alkyl acrylate/hydroxyethylacrylate copolymer and allyl stearate/vinyl acetate (VA) copolymer.

### BACKGROUND

Cosmetic formulations on the market often provide excellent appearance in terms of color and uniformity. However, they are often devoid of skin care ingredients due to disruption of the stability and oftentimes, adding such ingredients compromises the sensory of the product.

Indeed, it is a challenge to incorporate many skin-beneficial ingredients and actives into such formulas while also controlling overall stability of the finalized formula. These types of products also require certain cosmetic and sensorial characteristics, including, e.g., a non-tacky feel, and/or a hydrating formula that provides, e.g., a shiny, matte, or satin appearance to lips.

### BRIEF SUMMARY

A cosmetic composition may be provided. The composition may be an emulsion. The cosmetic composition may include a C10-C40 alkyl acrylate polymer, such as a C12-22 alkyl acrylate/hydroxyethylacrylate copolymer. The cosmetic composition may include an allylic polymer, such as allyl stearate/VA copolymer. The cosmetic composition may include an emulsifying agent or stabilizer, such as Polyglyceryl-4 Diisostearate/Polyhydroxystearate/Sebacate.

The composition may include a polar solvent. The polar solvent may be a polyol. The polyol may be glycerin and may optionally include a glyceryl ether and/or glyceryl ester.

The composition may include one or more fatty substances. The fatty substance(s) may be present in a total amount of at least 30% by weight of the cosmetic composition. The fatty substance(s) may include, e.g., diisostearyl polyglyceryl-3 dimer dilinoleate.

The composition may include a polymeric elastomer. The composition may include a dispersing agent. The composition may include a hydrocarbon-based resin. The composition may include a polyolefin, colorant, preservative, skin care agent, and/or an antioxidant. The composition may be substantially free of water.

In various embodiments, a method may be provided. The method may include providing a cosmetic composition as disclosed herein. The method may include applying the cosmetic composition to a lip or skin.

### BRIEF DESCRIPTION OF DRAWINGS

FIG. 1 is a graph showing the results of texture analyses of several formulations, including one exemplary formulation and two comparative formulas.

### DETAILED DESCRIPTION

As used herein, articles such as “a” and “an” when used in a claim, are understood to mean one or more of what is claimed or described.

As used herein, the expression “at least one” means one or more and thus includes individual components as well as mixtures/combinations.

All percentages listed are by weight unless otherwise noted. All concentrations are by weight percent on an active basis unless otherwise indicated.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “between” two number is intended to be inclusive of the bounding numbers. Thus, between 0 and 1 would include both 0 and 1.

As used herein, the terms “include”, “includes” and “including” are meant to be non-limiting.

As used herein, the term “substantially free [of an ingredient]” means that the composition contains less than 1% of the identified ingredient. The term “free [of an ingredient]” means the composition contains less than 0.1% of the identified ingredient, preferably below detectable amounts of the identified ingredient, and most preferably none of the identified ingredient.

The disclosed formulas combine a C10-C40 alkyl acrylate polymer and an allylic polymer to provide a unique experience. The combination surprisingly improves the stability of emulsions, such as glycerin emulsions, while maintaining a low tack sensory feel.

Thus, a cosmetic composition may be provided. The composition may be an emulsion.

### Polymers.

The cosmetic composition may include two types of polymers.

The cosmetic composition may include a C10-C40 alkyl acrylate polymer. The C10-C40 alkyl acrylate polymer may be a homopolymer. The C10-C40 alkyl acrylate polymer may be a copolymer. Non-limiting examples of such polymers include Acrylates/C12-22 Alkyl Methacrylate Copolymer, Acrylates/C12-22 Alkyl Methacrylate Copolymer, C12-22 Alkyl Acrylates/Hydroxyethylacrylate Copolymer, and/or acrylates/C10-C30 alkyl acrylate crosspolymers. In some embodiments, the C1-C40 alkyl acrylate polymer may be, e.g., TEGO® SP-6 (Poly C10-30 Alkyl Acrylate), TEGO® SP 13-1 (Poly C10-30 Alkyl Acrylate), and/or TEGO® SP 13-6 (Poly C10-30 Alkyl Acrylate). In some embodiments, the C10-C40 alkyl acrylate polymer may be, e.g., a modified acrylate homopolymer, such as a stearyl- or behenyl-modified acrylate homopolymer

The C10-C40 alkyl acrylate polymer may be present in an amount no more than 10% by weight of the composition. The C10-C40 alkyl acrylate polymer may be present in an amount no more than 5% by weight of the composition. In some embodiments, the C10-C40 alkyl acrylate polymer may be present in an amount no more than 4% by weight of the composition. In some embodiments, the C10-C40 alkyl acrylate polymer may be present in an amount no more than 3% by weight of the composition. In some embodiments, the C10-C40 alkyl acrylate polymer may be present in an amount no more than 2.5% by weight of the composition. In some embodiments, the C10-C40 alkyl acrylate polymer may be present in an amount of at least 0.5% by weight of the composition. In some embodiments, the C10-C40 alkyl acrylate polymer may be present in an amount of at least 1% by weight of the composition. In some embodiments, the C10-C40 alkyl acrylate polymer may be present in an amount of at least 1.5% by weight of the composition.

The cosmetic composition may include an allylic polymer. The term “allylic,” as used herein, describes an alkenyl group wherein a carbon-carbon double bond is connected to a methylene group, and the methylene group represents the point of attachment to the rest of the molecule. Non-limiting example of allylic polymers include allyl stearate/vinyl acetate (VA) copolymer, allyl 2,2-dimethylpentanoate/vinyl laurate, allyl dimethylpropionate/vinyl stearate, and allyl propionate/allyl stearate.

The allylic polymer may be present in an amount no more than 10% by weight of the composition. The allylic polymer may be present in an amount no more than 5% by weight of the composition. In some embodiments, the allylic polymer may be present in an amount no more than 4% by weight of the composition. In some embodiments, the allylic polymer may be present in an amount no more than 3% by weight of the composition. In some embodiments, the allylic polymer may be present in an amount no more than 2.5% by weight of the composition. In some embodiments, the allylic polymer may be present in an amount of at least 0.5% by weight of the composition. In some embodiments, the allylic polymer may be present in an amount of at least 1% by weight of the composition. In some embodiments, the allylic polymer may be present in an amount of at least 1.5% by weight of the composition.

### Emulsifying Agent or Stabilizer:

The cosmetic composition may include a emulsifying agent or stabilizer. The emulsifying agent or stabilizer may be present in a total amount of no more than 10% by weight of the composition. The emulsifying agent or stabilizer may be present in a total amount of no more than 8% by weight of the composition. In some embodiments, the emulsifying agent or stabilizer may be present in a total amount of no more than 4.5% by weight of the composition. In some embodiments, the emulsifying agent or stabilizer may be present in a total amount of no more than 4% by weight of the composition. In some embodiments, the emulsifying agent or stabilizer may be present in a total amount of no more than 3.5% by weight of the composition. In some embodiments, the emulsifying agent or stabilizer may be present in a total amount of at least 1% by weight of the composition. In some embodiments, the emulsifying agent or stabilizer may be present in a total amount of at least 1.5% by weight of the composition. In some embodiments, the emulsifying agent or stabilizer may be present in a total amount of at least 2% by weight of the composition. In some embodiments, the emulsifying agent or stabilizer may be present in a total amount of at least 2.5% by weight of the composition.

The emulsifying agent or stabilizer may comprise or consist of a fatty acid ester of a polyol. The fatty acid esters may be esters of isostearic acid and glycerol, such as for example polyglycerolated (4 moles) isostearate (INCI name: Polyglyceryl-4 Isostearate) sold under the name Isolan GI34® by Evonik, polyglycerolated (3 mol) diisostearate sold under the name Lameform TGI® by BASF; polyglycerolated distearate (2 mol) sold under the name Emalex PGSA® by Nihon emulsion; polyglycerolated (10 mol) monoisostearate sold under the name Nikkol Decaglyn 1-IS® by Nihon Surfactant (INCI name: Polyglyceryl-10 isostearate); and polyglyceryl-4 diisostearate/polyhydroxystearate/sebacate sold under the name Isolan GPS® by Evonik. In a preferred embodiment, the only emulsifying agent or stabilizer is polyglyceryl-4 diisostearate/polyhydroxystearate/sebacate.

### Polar Solvent.

The composition may include a polar solvent. The polar solvent may be an organic polar solvent. The organic polar solvent may be liquid at room temperature (25° C.) and at least partially water-miscible. Non-limiting examples include alkanols such as ethyl alcohol, isopropyl alcohol, aromatic alcohols such as benzyl alcohol and phenylethyl alcohol, or polyols or polyol ethers, for instance ethylene glycol monomethyl, monoethyl or monobutyl ether, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether.

The polar solvent may include water. In some embodiments, the composition may be free of water. In some embodiments, the composition may be substantially free of water. In some embodiments, water may be present in a total amount no more than 5% by weight of the composition. In some embodiments, water may be present in a total amount no more than 10% by weight of the composition. In some embodiments, water may be present in a total amount no more than 15% by weight of the composition. In some embodiments, water may be present in a total amount no more than 20% by weight of the composition. In some embodiments, water may be present in a total amount no more than 30% by weight of the composition. In some embodiments, water may be present in a total amount no more than 40% by weight of the composition. In some embodiments, water may be present in a total amount no more than 50% by weight of the composition. In some embodiments, water may be present in a total amount no more than 60% by weight of the composition.

The polar solvent may comprise or consist of a polyol. Non-limiting examples of polyols include ethylene glycol, propylene glycol such as 1,2-propylene glycol and 1,3-propylene glycol, glycerol, pentaerythritol, trimethylolpropane, 1,4,6-octanetriol, butanediol, pentanediol, hexanediol, dodecanediol, octanediol, chloropentanediol, glycerol monoallyl ether, glycerol monoethyl ether, diethylene glycol, 2-ethylhexanediol-1,4, cyclohexanediol-1,4, 1,2,6-hexanetriol, neopental glycol, 1,3,5-hexanetriol, 1,3-bis-(2-hydroxyethoxy)propane and the like. In some embodiments, the polyol may comprise or consist of glycerin. The polyol may comprise or consist of glycerin and one or more glyceryl ethers or glyceryl esters. Non-limiting examples of glyceryl ethers include, e.g., alkyl glyceryl ethers such as isostearyl glyceryl ether, and ethylhexyl glycerin. Non-limiting examples of glyceryl esters includes glyceryl mono fatty acid esters such as glyceryl caprylate, glyceryl caprate, glyceryl laurate, glyceryl myristate, glyceryl stearate, glyceryl linoleate, glyceryl oleate, glyceryl isostearate, and glyceryl behenate.

In some embodiments, the polar solvent may be a water-soluble solvent. In some embodiments, the polar solvent may be a water-miscible solvent. In some embodiments, the polar solvent may include both a water-soluble solvent and a water-miscible solvent. As used herein, the term “water-soluble solvent” refers to compounds that are liquid at 25° C. and at atmospheric pressure (760 mmHg), and it has a solubility of at least 70% in water under these conditions. In some cases, the water-soluble solvent has a solubility of at least 80%, or at least 90%. As used herein, the term “water-miscible solvent” refers to compounds that are liquid at 25° C. and at atmospheric pressure (760 mmHg), and it has a solubility of at least 50′% in water under these conditions. In some cases, the water-miscible solvent has a solubility of at least 60% in water under these conditions.

The polar solvent may be present in a total amount of not more than 20% by weight of the composition. The polar solvent may be present in a total amount of not more than 15% by weight of the composition. The polar solvent may be present in a total amount of not more than 10% by weight of the composition. The polar solvent may be present in a total amount of not more than 9% by weight of the composition. The polar solvent may be present in a total amount of not more than 8% by weight of the composition. The polar solvent may be present in a total amount of not more than 7% by weight of the composition. The polar solvent may be present in a total amount of not more than 6% by weight of the composition. The polar solvent may be present in a total amount of at least 1% by weight of the composition. The polar solvent may be present in a total amount of at least 2% by weight of the composition. The polar solvent may be present in a total amount of at least 3% by weight of the composition. The polar solvent may be present in a total amount of at least 4% by weight of the composition. The polar solvent may be present in a total amount of at least 5% by weight of the composition.

### Fatty Substances.

The composition may include one or more fatty substances. The fatty substance(s) may include a hydrocarbon other than a hydrocarbon-based resin. The fatty substances may include an emollient. Non-limiting examples of such emollients include, e.g., squalene, plant derived oils, esters such as hydrocarbon-based esters, adipate esters, aromatic esters, pentaerythritol esters, dimer dilinoleate esters, fatty acid triglycerides, or a combination thereof.

The fatty substance(s) may be present in a total amount of at least 30% by weight of the cosmetic composition. The fatty substance(s) may be present in a total amount of at least 35% by weight of the cosmetic composition. The fatty substance(s) may be present in a total amount of at least 40% by weight of the cosmetic composition. The fatty substance(s) may be present in a total amount of at least 45% by weight of the cosmetic composition. The fatty substance(s) may be present in a total amount of at least 50% by weight of the cosmetic composition. The fatty substance(s) may be present in a total amount of at least 55% by weight of the cosmetic composition. The fatty substance(s) may be present in a total amount of at least 60% by weight of the cosmetic composition. The fatty substance(s) may be present in a total amount of at least 65% by weight of the cosmetic composition. The fatty substance(s) may be present in a total amount of at least 70% by weight of the cosmetic composition. The fatty substance(s) may be present in a total amount of no more than 90% c by weight of the cosmetic composition. The fatty substance(s) may be present in a total amount of no more than 85% by weight of the cosmetic composition. The fatty substance(s) may be present in a total amount of no more than 80% by weight of the cosmetic composition. The fatty substance(s) may be present in a total amount of no more than 75% by weight of the cosmetic composition.

Fatty Acid Esters of Glycerol and/or Polyglycerol.

The fatty substance may include one or more fatty acid esters of glycerol or polyglycerol. The fatty acid ester (s) of polyglycerol may be esters resulting from the reaction of polyglycerol comprising from 2 to 12 glycerol units, preferably from 3 to 10 glycerol units, and of at least one fatty acid containing from 8 to 24 carbon atoms, preferably from 8 to 22 carbon atoms, better still from 10 to 20 carbon atoms and even better still from 10 to 18 carbon atoms. The fatty acids may include from 8 to 24 carbon atoms may be linear or branched, and saturated or unsaturated. The fatty acids may be oleic acid, stearic acid, isostearic acid, lauric acid, palmitic acid, myristic acid, linoleic acid, capric acid and caprylic acid, and/or mixtures thereof.

The fatty acid esters of polyglycerol may be monoesters, diesters, triesters, tetraesters, polyesters, and/or mixtures thereof. The esters may have a low degree of esterification, for instance fatty acid monoesters, diesters or triesters of polyglycerol, or a mixture. The fatty acid ester of polyglycerol may be in the form of a mixture of esters with a low degree of esterification, for instance a mixture of monoester and diester or a mixture of monoester, diester and triester.

Non-limiting examples of fatty acid esters of glycerol or polyglycerol include: polyglyceryl-2 distearate; polyglyceryl-10 decastearate; glyceryl oleate; glyceryl stearate; polyglyceryl-5 hexastearate; polyglyceryl-10 pentaoleate; polyglyceryl-10 pentastearate: glyceryl caprylate/caprate; polyglyceryl-10 heptaoleate; polyglyceryl-4 isostearate; diisostearoyl polyglyceryl-3 dimer dilinoleate; glyceryl laurate; polyglyceryl-5 trioleate: polyglyceryl-2 oleate; polyglyceryl-5 trimyristate; polyglyceryl-2 caprylate; and/or polyglyceryl-2 laurate.

In some embodiments, the fatty acid ester(s) of glycerol or polyglycerol may be present in a total amount that is no more than about 5% by weight of the composition. In some embodiments, the fatty acid ester(s) of glycerol or polyglycerol may be present in a total amount that is no more than about 4% by weight of the composition. In some embodiments, the fatty acid ester(s) of glycerol or polyglycerol may be present in a total amount that is no more than about 3% by weight of the composition. In some embodiments, the fatty acid ester(s) of glycerol or polyglycerol may be present in a total amount that is no more than about 2% by weight of the composition. In some embodiments, the fatty acid ester(s) of glycerol or polyglycerol may be present in a total amount that is no more than about 1% by weight of the composition. In some embodiments, the composition may be substantially free of fatty acid ester(s) of glycerol or polyglycerol.

### Dilinoleate Ester Dimer.

In some embodiments, the fatty substances may include a dilinoleate ester dimer. Non-limiting examples of such dilinoleate ester dimers include: bis-behenyl/isostearyl/phytosteryl dimer dilinoleyl dimer dilinoleate; phytosteryl isostearyl dimer dilinoleate; phytosteryl dilinoleate/isostearyl dimer dilinoleate cetyl/stearyl/behenyl; and/or diisostearoyl polyglyceryl-3 dimer dilinoleate.

In some embodiments, the dilinoleate ester dimer(s) may be present in a total amount that is no more than about 30% by weight of the composition. In some embodiments, the dilinoleate ester dimer(s) may be present in a total amount that is no more than about 25% by weight of the composition. In some embodiments, the dilinoleate ester dimer(s) may be present in a total amount that is no more than about 20% by weight of the composition. In some embodiments, the dilinoleate ester dimer(s) may be present in a total amount that is at least 5% by weight of the composition. In some embodiments, the dilinoleate ester dimer(s) may be present in a total amount that is at least 10% by weight of the composition. In some embodiments, the dilinoleate ester dimer(s) may be present in a total amount that is at least 15% by weight of the composition. In some embodiments, the composition may be free, or substantially free, of dilinoleate ester dimer(s).

In a preferred embodiment, the fatty substance(s) include diisostearoyl polyglyceryl-3 dimer dilinoleate and bis-behenyl/isostearyl/phytosteryl dimer dilinoleyl dimer dilinoleate.

### Triesters Containing at Least 35 Carbon Atoms.

The fatty substance(s) may include a triester containing at least 35 carbon atoms, such as between 35 and 70 carbon atoms in total. Non-limiting examples of such triesters include triesters of a tricarboxylic acid, such as triisostearyl citrate, or tridecyl trimellitate, or glycol triesters of monocarboxylic acids such as polyglyceryl-2 triisostearate. In a preferred embodiment, the triester is tridecyl trimellitate.

In some embodiments, the triester(s) may be present in a total amount that is no more than about 30% by weight of the composition. In some embodiments, the triester(s) may be present in a total amount that is no more than about 25% by weight of the composition. In some embodiments, the triester(s) may be present in a total amount that is no more than about 20% by weight of the composition. In some embodiments, the triester(s) may be present in a total amount that is at least 5% by weight of the composition. In some embodiments, the triester(s) may be present in a total amount that is at least 10% by weight of the composition. In some embodiments, the triester(s) may be present in a total amount that is at least 15% by weight of the composition. In some embodiments, the composition may be free, or substantially free, of triester(s).

Linear or Branched Hydrocarbons with More than 16 Carbon Atoms.

The fatty substance(s) may include a linear or branched hydrocarbons of mineral, animal or synthetic origin with more than 16 carbon atoms. Non-limiting examples include liquid paraffins, liquid petroleum jelly, polydecenes, polybutene, hydrogenated polyisobutene, and squalane.

In some embodiments, the linear or branched hydrocarbon(s) may be present in a total amount that is no more than about 40% by weight of the composition. In some embodiments, the linear or branched hydrocarbon(s) may be present in a total amount that is no more than about 35% by weight of the composition. In some embodiments, the linear or branched hydrocarbon(s) may be present in a total amount that is no more than about 30% by weight of the composition. In some embodiments, the linear or branched hydrocarbon(s) may be present in a total amount that is at least 5% by weight of the composition. In some embodiments, the linear or branched hydrocarbon(s) may be present in a total amount that is at least 10% by weight of the composition. In some embodiments, the linear or branched hydrocarbon(s) may be present in a total amount that is at least 15% by weight of the composition. In some embodiments, the linear or branched hydrocarbon(s) may be present in a total amount that is at least 20% by weight of the composition. In some embodiments, the linear or branched hydrocarbon(s) may be present in a total amount that is at least 25% by weight of the composition. In some embodiments, the composition may be free, or substantially free, of linear or branched hydrocarbon(s).

In some embodiments, the composition may include polybutene in an amount from about 5% to about 20% by weight of the composition. In some embodiments, the polybutene may be present in a total amount of at least 4%. In some embodiments, the polybutene may be present in a total amount of at least 5%. In some embodiments, the polybutene may be present in a total amount of at least 6%. In some embodiments, the polybutene may be present in a total amount of at least 7%. In some embodiments, the polybutene may be present in a total amount of at least 8%. In some embodiments, the polybutene may be present in a total amount of at least 9%. In some embodiments, the polybutene may be present in a total amount of at least 10%. In some embodiments, the polybutene may be present in a total amount of no more than 20%. In some embodiments, the polybutene may be present in a total amount of no more than 19%. In some embodiments, the polybutene may be present in a total amount of no more than 18%. In some embodiments, the polybutene may be present in a total amount of no more than 17%. In some embodiments, the polybutene may be present in a total amount of no more than 16%. In some embodiments, the polybutene may be present in a total amount of no more than 15%.

Triglycerides of Saturated or Unsaturated C₈-C₂₀ Fatty Acids.

The fatty substance(s) may include a Triglycerides of a saturated or unsaturated C₅-C₂₀ fatty acid. Non-limiting examples include caprylic/capric triglyceride, glyceryl triheptanoate, glyceryl trioctanoate, or glyceryl triisostearate.

In some embodiments, the triglyceride(s) may be present in a total amount that is no more than about 5% by weight of the composition. In some embodiments, the triglyceride(s) may be present in a total amount that is no more than about 4% by weight of the composition. In some embodiments, the triglyceride(s) may be present in a total amount that is no more than about 3% by weight of the composition. In some embodiments, the triglyceride(s) may be present in a total amount that is no more than about 2% by weight of the composition. In some embodiments, the triglyceride(s) may be present in a total amount that is no more than about 1% by weight of the composition. In some embodiments, the composition may be substantially free of triglyceride(s). In some embodiments, the composition may be free, or substantially free, of the triglyceride(s).

### Plant-Derived Oils.

The fatty substance(s) may include a plant-derived oil. Non-limiting examples include Zea mays (corn) oil, brassica campestris (rapeseed) seed oil, Butyrospermum parkii (shea) butter, Prunus amygdalus dulcis (sweet almond) oil, Prunus armeniaca (apricot) kernel oil, and Helianthus anmus (sunflower) seed oil, Calophyllum, palm, Ricinus communis (castor), Elaeis guineensis (palm), avocado oil, Simmondsia chinensis (jojoba) seed oil, Olea europaea (olive) oil, Cocos mucifera (coconut) oil, and cereal germ oil, caprylic/capric triglyceride, Sesamum indicum (sesame) seed oil, Triticum vulgare (wheat) germ oil, argania spinose kernel oil, Glycine soja (soybean) oil, canola oil, Oryza sativa (rice) bran oil, Limnanthes alba (meadowfoam) seed oil, and combinations thereof.

In some embodiments, the plant derived oil(s) may be present in a total amount that is no more than about 2% by weight of the composition. In some embodiments, the plant derived oil(s) may be present in a total amount that is no more than about 1.5% by weight of the composition. In some embodiments, the plant derived oil(s) may be present in a total amount that is no more than about 1% by weight of the composition. In some embodiments, the plant derived oil(s) may be present in a total amount that is no more than about 0.5% by weight of the composition. In some embodiments, the composition may be substantially free of plant derived oil(s).

### Monoesters of Monoalcohols.

The fatty substance(s) may include a monoester of a monoalcohol. Non-limiting examples include dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononate; octyldodecyl eructate; oleyl eructate; ethyl palmitate, isopropyl palmitate, 2-ethylhexyl palmitate, 2-octyldecyl palmitate, alkyl myristates such as isopropyl 2-octyldodecyl myristate, isobutyl stearate; 2-hexyldecyl laurate, and mixtures thereof.

In some embodiments, the monoester(s) may be present in a total amount that is no more than about 10% by weight of the composition. In some embodiments, the monoester(s) may be present in a total amount that is no more than about 8% by weight of the composition. In some embodiments, the monoester(s) may be present in a total amount that is no more than about 6% by weight of the composition. In some embodiments, the monoester(s) may be present in a total amount that is at least about 1% by weight of the composition. In some embodiments, the monoester(s) may be present in a total amount that is at least about 3% by weight of the composition. In some embodiments, the monoester(s) may be present in a total amount that is at least about 5% by weight of the composition. In some embodiments, the composition may be free, or substantially free, of monoester(s).

### Pentaerythritol Esters.

The fatty substance(s) may include a pentaerythritol ester, such as pentaerythrityl tetraisostearate.

In some embodiments, the pentaerythritol ester(s) may be present in a total amount that is no more than about 10% by weight of the composition. In some embodiments, the pentaerythritol ester(s) may be present in a total amount that is no more than about 8% by weight of the composition. In some embodiments, the pentaerythritol ester(s) may be present in a total amount that is no more than about 6% by weight of the composition. In some embodiments, the pentaerythritol ester(s) may be present in a total amount that is at least about 1% by weight of the composition. In some embodiments, the pentaerythritol ester(s) may be present in a total amount that is at least about 2% by weight of the composition. In some embodiments, the pentaerythritol ester(s) may be present in a total amount that is at least about 3% by weight of the composition. In some embodiments, the pentaerythritol ester(s) may be present in a total amount that is at least about 4% by weight of the composition. In some embodiments, the composition may be free, or substantially free, of pentaerythritol ester(s).

### Alkylene Carbonate.

The fatty substance(s) may include an alkylene carbonate. Non-limiting examples include C4-C6 cyclic carbonates, particularly selected from the group consisting of propylene carbonate, dipropylene carbonate, butylene carbonate, 2,3-butylene carbonate, 2,3-pentylene carbonate, pentylene carbonate, ethylene carbonate. Preferably, the at least one C4-C6 cyclic carbonate is propylene carbonate.

In some embodiments, the alkylene carbonate(s) may be present in a total amount that is no more than about 2% by weight of the composition. In some embodiments, the alkylene carbonate(s) may be present in a total amount that is no more than about 1.5% by weight of the composition. In some embodiments, the alkylene carbonate(s) may be present in a total amount that is no more than about 1% by weight of the composition. In some embodiments, the alkylene carbonate(s) may be present in a total amount that is no more than about 0.5% by weight of the composition. In some embodiments, the composition may be free, or substantially free, of alkylene carbonate(s).

### Polymeric Elastomer.

The composition may include a polymeric elastomer.

The polymeric elastomer may be a styrenic block copolymer elastomer.

The styrenic block copolymers of the present invention are characterized by the presence of at least one “hard” segment, and at least one “soft” segment. Aside from their compositional nature, the hard and soft segments of the block copolymers of the present invention may be defined in terms of their respective glass transition temperatures, T_{g}. The hard segment may have a T_{g} of 50° C. or more, whereas the soft segment may have a T_{g} of 20° C. or less. The T_{g} for the hard block can range from 50° C. to 150° C. The T_{g} for the soft block can range from −150° C. to 20° C.

Block copolymers useful in compositions of the present invention may be thermoplastic elastomers. The hard segments of the thermoplastic elastomer typically comprise vinyl monomers in varying amounts. Examples of suitable vinyl monomers include, but are not limited to, styrene as well as other optional monomers including methacrylate, acrylate, vinyl ester, vinyl ether, vinyl acetate, and the like. At least one hard segment should include styrene.

The soft segments of the thermoplastic elastomer comprise olefin polymers and/or copolymers which may be saturated, unsaturated, or combinations thereof. Suitable olefin copolymers may include, but are not limited to, ethylene/propylene copolymers, ethylene/butylene copolymers, propylene/butylene copolymers, polybutylene, polyisoprene, polymers of hydrogenated butanes and isoprenes, and mixtures thereof.

Thermoplastic elastomers useful in the present invention are block copolymers, e.g., di-block, tri-block, multi-block, radial and star block copolymers, and mixtures and blends thereof. A di-block thermoplastic elastomer is usually defined as an A-B type or a hard segment (A) followed by a soft segment (B) in sequence. A tri-block is usually defined as an A-B-A type copolymer or a ratio of one hard, one soft, and one hard segment. Multi-block or radial block or star block thermoplastic elastomers usually contain any combination of hard and soft segments, provided that the elastomers possess both hard and soft characteristics.

In some embodiments, the thermoplastic elastomer of the present invention may be chosen from the class of KRATON rubbers (Kraton Corporation of Houston, Tex.) or from similar thermoplastic elastomers. KRATON rubbers are thermoplastic elastomers in which the polymer chains comprise a di-block, tri-block, multi-block or radial or star block configuration or numerous mixtures thereof. The KRATON tri-block rubbers have polystyrene (hard) segments on each end of a rubber (soft) segment, while the KRATON di-block rubbers have a polystyrene (hard) segment attached to a rubber (soft) segment. The KRATON radial or star configuration may be a four-point or other multipoint star made of rubber with a polystyrene segment attached to each end of a rubber segment. The configuration of each of the KRATON rubbers forms separate polystyrene and rubber domains.

Each molecule of KRATON rubber is said to comprise block segments of styrene monomer units and rubber monomer and/or co-monomer units. The most common structure for the KRATON triblock copolymer is the linear A-B-A block type styrene-butadiene-styrene, styrene-isoprene-styrene, styrene-ethylenepropylene-styrene, or styrene-ethylene-butylene-styrene. The KRATON di-block is preferably the AB block type such as styrene-ethylene-propylene, styrene-ethylene-butylene, styrene-butadiene, or styrene-isoprene. The KRATON rubber configuration is well known in the art and any block copolymer elastomer with a similar configuration is within the practice of the invention. Other block copolymers are sold under the tradename Septon (which represent elastomers known as SEEPS, sold by Kurary, Co., Ltd) and those sold by ExxonMobil Chemical under the tradename VECTOR.

Other thermoplastic elastomers useful in the present invention may include those block copolymer elastomers comprising a styrene-butylene/ethylene-styrene copolymer (tri-block), an ethylene/propylene-styrene copolymer (radial or star block) or a mixture or blend of the two. (Some manufacturers refer to block copolymers as hydrogenated block copolymers, e.g. hydrogenated styrene-butylene/ethylene-styrene copolymer (tri-block)).

Compositions may include at least one block copolymer, e.g., diblock, triblock, multiblock or radial block copolymers, and mixtures thereof. The at least one block copolymer may include at least one styrene block and may further comprise at least one block comprising units chosen from butadiene, ethylene, propylene, butylene and isoprene or a mixture thereof.

Diblock copolymers that may be mentioned include but are not limited to styrene/ethylene-propylene copolymers (comprising a styrene block and a block obtained from ethylene and propylene), styrene/ethylene-butylene copolymers, styrene-ethylene/butadiene copolymers, styrene/butadiene copolymers and styrene/isoprene copolymers.

In certain notable embodiments, the block copolymer includes styrene blocks and one or more blocks selected from: butadiene blocks, isoprene blocks, and ethylene-butylene blocks. In other embodiments, the block copolymer includes (1) styrene blocks and butadiene blocks; or (2) styrene and ethylene-butadiene blocks; or (3) styrene and isoprene blocks.

Triblock copolymers that may be mentioned include but are not limited to styrene/ethylene-propylene/styrene copolymers, styrene/ethylene-butylene/styrene copolymers, styrene/ethylene-butadiene/styrene copolymers, styrene/isoprene/styrene copolymers and styrene/butadiene/styrene copolymers.

In some embodiments, the composition may include a mixture of a diblock copolymer and a triblock copolymer. copolymer. According to at least one embodiment, the diblock copolymer and the triblock copolymer may be chosen from block copolymers comprising at least one styrene block and at least one block comprising units chosen from butadiene, ethylene, propylene, butylene and isoprene. In one embodiment, the block copolymer has from about 50% to about 90% triblock and from about 10% to about 50% diblock.

In some embodiments, the composition may include a butylene/ethylene/styrene copolymer or an ethylene/propylene/styrene copolymer. In some embodiments, the composition may include both a butylene/ethylene/styrene copolymer and an ethylene/propylene/styrene copolymer.

The polymeric elastomer may be a silicone elastomer.

The silicone elastomer may be a non-emulsifying silicone elastomer. The term “non-emulsifying” defines organopolysiloxane elastomers not containing any hydrophilic chains, and in particular not containing any polyoxyalkylene units (especially polyoxyethylene or polyoxypropylene) or any polyglyceryl units. Thus, according to one particular mode of the invention, the composition comprises an organopolysiloxane elastomer free of polyoxyalkylene units and of polyglyceryl units.

Non-limiting examples of the silicone elastomer include Dimethicone Crosspolymer (INCI name), Vinyl Dimethicone Crosspolymer (INCI name), Dimethicone/Vinyl Dimethicone Crosspolymer (INCI name), Dimethicone Crosspolymer-3 (INCI name).

The silicone elastomer may be a self-emulsifying silicone elastomer. Non-limiting examples of self-emulsifying silicone elastomers include, but are not limited to, substituted or unsubstituted dimethicone/copolyol crosspolymer, dimethicone (and) dimethicone/vinyldimethicone crosspolymer, dimethicone and dimethicone/PEG-10/15 crosspolymers, substituted or unsubstituted dimethicone/polyglyceral crosspolymer, dimethicone and dimethicone/polyglycerin-3 crosspolymer.

The polymeric elastomer may be present in a total amount of not more than 3% by weight of the composition. The polymeric elastomer may be present in a total amount of not more than 2% by weight of the composition. The polymeric elastomer may be present in a total amount of not more than 1% by weight of the composition. The polymeric elastomer may be present in a total amount of not more than 0.5% by weight of the composition. In some embodiments, the composition may be free, or substantially free, of styrenic block copolymers. In some embodiments, the composition may be free, or substantially free, of non-emulsifying silicone elastomers. In some embodiments, the composition may be free, or substantially free, of self-emulsifying silicone elastomers.

### Dispersing Agent.

The composition may include a dispersing agent. The dispersing agent serves to protect the dispersed particles against agglomeration or flocculation thereof. The dispersing agent may physically or chemically attach to the surface of the pigments. These dispersants may also contain at least one functional group that is compatible with or soluble in the continuous medium. In particular, 12-hydroxystearic acid esters and in particular 08 to 020 fatty acid esters of polyols such as glycerol or digylcerol may be used, such as poly(12-hydroxystearic acid) stearate with a molecular weight of about 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

The dispersing agent may be present in a total amount of not more than 10% by weight of the composition. The dispersing agent may be present in a total amount of not more than 9% by weight of the composition. The dispersing agent may be present in a total amount of not more than 8% by weight of the composition. The dispersing agent may be present in a total amount of not more than 7% by weight of the composition. The dispersing agent may be present in a total amount of not more than 6% by weight of the composition. The dispersing agent may be present in a total amount of at least 1% by weight of the composition. The dispersing agent may be present in a total amount of at least 2% by weight of the composition. The dispersing agent may be present in a total amount of at least 3% by weight of the composition. The dispersing agent may be present in a total amount of at least 4% by weight of the composition. The dispersing agent may be present in a total amount of at least 5% by weight of the composition.

### Hydrocarbon-Based Resin.

The composition may include a hydrocarbon-based resin. The hydrocarbon-based resin may be a hydrocarbon-based film former.

The composition according to the invention may include at least one hydrocarbon-based resin with a number-average molecular weight of less than or equal to 10 000 g/mol. The number-average molecular weight may range from 250 g/mol to 10 000 g/mol, preferably from 250 g/mol to 5000 g/mol, more preferentially from 250 g/mol to 2000 g/mol, or even from 250 g/mol to 1000 g/mol. The number-average molecular weights (Mn) may be determined by gel permeation liquid chromatography (THF solvent, calibration curve established with linear polystyrene standards, refractometric detector).

The softening point of the resin ranges from 70 to 130° C., more particularly from 80 to 120° C. and preferably from 90 to 110° C. (standard ASTM E 28).

The hydrocarbon-based resin may be an aliphatic hydrocarbon-based resin. Non-limiting examples of aliphatic hydrocarbon-based resins include:
hydrogenated indene/methylstyrene/styrene copolymers, for example sold under the name Regalite by the company Eastman Chemical, in particular Regalite R1090, Regalite R1100, Regalite S1100 and Regalite S5100, or under the name Arkon P-90, Arkon P-100, and Arkon P-115 by the company Arakawa;
aliphatic pentanediene resins especially such as those derived from the majority polymerization of the 1,3-pentanediene (trans- or cis-piperylene) monomer and of minority monomers chosen from isoprene, butene, 2-methyl-2-butene, pentene and 1,4-pentanediene, and mixtures thereof. These resins may have a molecular weight ranging from 1000 to 2500 g/mol. Such 1,3-pentanediene resins are sold, for example, under the references Piccotac 95 by the company Eastman Chemical, Escorez 1102, Escorez 1304, Escorez 1310LC and Escorez 1315 by the company Exxon Chemicals, or Wingtack 95 by the company Cray Valley;
diene resins of cyclopentanediene dimers, for instance those derived from the polymerization of dicyclopentanediene, methyldicyclopentanediene and other pentanediene dimers, and mixtures thereof. These resins generally have a molecular weight ranging from 500 to 800 g/mol, for instance those sold under the reference Escorez 5380, Escorez 5300, Escorez 5400, Escorez 5415 or Escorez 5490 by the company ExxonMobil Chem., and the resins Sukorez SU-90, Sukorez SU-100, Sukorez SU-110, Sukorez SU-100S, Sukorez SU-200, Sukorez SU-210, Sukorez SU-490 and Sukorez SU-400, by the company Kolon;
hydrogenated resins derived from the polymerization of pentanediene, such as, inter alia, those sold under the name Eastotac H-100E, Eastotac H-115E, Eastotac C-100L, Eastotac C-115L, Eastotac H-100L, Eastotac H-115L, Eastotac C-100R, Eastotac C-115R, Eastotac H-100R, Eastotac H-115R, Eastotac C-100W, Eastotac C-115W, Eastotac H-100W and Eastotac H-115W, by the company Eastman Chemical Co.;
and mixtures thereof.

One such resin may be, the hydrogenated indene/methylstyrene/styrene copolymer sold especially under the name Regalite® R1110 resin by the company Eastman Chemical, or the aliphatic 1,3-pentanediene resin sold under the name Piccotac® 1095 resin by the company Eastman Chemical.

Preferably, the content of resin according to the invention is from 2% to 20% by weight, more particularly from 2% to 18% by weight and preferably from 3% to 16% by weight relative to the total weight of the composition.

The hydrocarbon-based resin may be present in a total amount of not more than 6% by weight of the composition. The hydrocarbon-based resin may be present in a total amount of not more than 5% by weight of the composition. The hydrocarbon-based resin may be present in a total amount of not more than 4% by weight of the composition. The hydrocarbon-based resin may be present in a total amount of at least 1% by weight of the composition. The hydrocarbon-based resin may be present in a total amount of at least 2% by weight of the composition. The hydrocarbon-based resin may be present in a total amount of at least 3% by weight of the composition.

### Polyolefin.

The composition may include a polyolefin. The polyolefin may be a polyolefin wax. As used herein, the term “wax” is intended to mean lipophilic compounds, which are solid at ambient temperature (20° C.) and at atmospheric pressure (760 mmHg), with a reversible solid/liquid change of state, which have a melting point of greater than or equal to 40° C., which may range up to 120° C. In some embodiments, the polyolefin waxes may have a melting point of at least about 80° C. In some embodiments, the polyolefin waxes may have a melting point of at least 80° C. The polyolefin waxes may be, e.g., homopolymers or copolymers of ethylene, of propene or butene, or even longer-chain α-olefins. In some embodiments, the polyolefin is polyethylene.

The polyolefin may be present in a total amount of not more than 5% by weight of the composition. The polyolefin may be present in a total amount of not more than 3% by weight of the composition. The polyolefin may be present in a total amount of not more than 2% by weight of the composition. The polyolefin may be present in a total amount of not more than 1.5% by weight of the composition. The polyolefin may be present in a total amount of not more than 1% by weight of the composition. The polyolefin may be present in a total amount of at least 0.1% by weight of the composition. The polyolefin may be present in a total amount of at least 0.25% by weight of the composition. The polyolefin may be present in a total amount of at least 0.5% by weight of the composition.

### Colorant.

The composition may include a colorant. In some embodiments, the colorant may be a pigment, a pearlescent agent, a goniochromatic pigment, a dye, or a combination thereof. In some embodiments, the composition can advantageously comprise at least one colorant chosen from pigments and/or pearlescent agents.

In some embodiments, the composition may include a total amount of colorant of no more than 15% by weight of the composition. In some embodiments, the composition may include a total amount of colorant of no more than 12.5% by weight of the composition. In some embodiments, the composition may include a total amount of colorant of no more than 10,% by weight of the composition. In some embodiments, the composition may include a total amount of colorant of no more than 7.5% by weight of the composition. In some embodiments, the composition may include a total amount of colorant of no more than 5% by weight of the composition. In some embodiments, the composition may include a total amount of colorant of no more than 4% by weight of the composition. In some embodiments, the composition may include a total amount of colorant of no more than 3% by weight of the composition. In some embodiments, the composition may include a total amount of colorant of no more than 2% by weight of the composition. In some embodiments, the composition may include a total amount of colorant of no more than 1% by weight of the composition. In some embodiments, the composition may be substantially free of colorant.

### Pigments.

As used herein, the term “pigment” refers to white or colored and inorganic (mineral) or organic particles which are insoluble in the lipophilic phase(s) and which are intended to color and/or opacify the composition and/or the deposited layer produced with the composition.

The pigments may be chosen from mineral pigments, organic pigments and composite pigments (i.e., pigments based on mineral and/or organic materials).

The pigments can be chosen from mineral pigments, in particular monochromatic pigments, organic lakes, pearlescent agents and goniochromatic pigments.

The mineral pigments can be chosen from metal oxide pigments, chromium oxides, iron oxides (black, yellow, red), titanium dioxide, zinc oxides, cerium oxides, zirconium oxides, chromium hydrate, manganese violet, Prussian blue, ultramarine blue, ferric blue, metal powders, such as aluminum powders or copper powder, and their mixtures.

Organic lakes are organic pigments, formed of a dye attached to a substrate. The lakes, which are also known as organic pigments, can be chosen from the materials below and their mixtures:
cochineal carmine;
organic pigments of azo dyes, anthraquinone dyes, indigoid dyes, xanthene dyes, pyrene dyes, quinoline dyes, triphenylmethane dyes or fluoran dyes.

Mention may in particular be made, among the organic pigments, of those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5 or FD&C Yellow No. 6;

the organic lakes can be insoluble sodium, potassium, calcium, barium, aluminum, zirconium, strontium or titanium salts of acid dyes, such as azo, anthraquinone, indigoid, xanthene, pyrene, quinoline, triphenylmethane or fluoran dyes, these dyes possibly comprising at least one carboxylic or sulfonic acid group.

The organic lakes can also be supported by an organic support, such as rosin or aluminum benzoate, for example.

Mention may in particular be made, among the organic lakes, of those known under the following names: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake or FD&C Yellow No. 6 Aluminum lake.

Mention may also be made of liposoluble dyes, such as, for example, Sudan Red, DC Red 17, DC Green 6, β-carotene, soybean oil, Sudan Brown, DC Yellow 11, DC Violet 2, DC Orange 5 or quinoline yellow.

The chemical substances corresponding to each of the organic colorants cited above are mentioned in the publication “International Cosmetic Ingredient Dictionary and Handbook”, 1997 edition, pages 371 to 386 and 524 to 528, published by The Cosmetic, Toiletries and Fragrance Association, the content of which is incorporated into the present patent application by reference.

The pigments may also have been subjected to a hydrophobic treatment.

Non-limiting examples of hydrophobic treatment agents include, e.g., silicones, such as methicones, dimethicones, alkoxysilanes and perfluoroalkylsilanes; fatty acids, such as stearic acid; metal soaps, such as aluminum dimyristate, the aluminum salt of hydrogenated tallow glutamate, perfluoroalkyl phosphates, perfluoroalkylsilanes, perfluoroalkylsilazanes, poly(hexafluoropropylene oxide)s, polyorganosiloxanes comprising perfluoroalkyl perfluoropolyether groups and amino acids; N-acylated amino acids or their salts; lecithin, isopropyl triisostearyl titanate, and their mixtures.

The N-acylated amino acids can comprise an acyl group having from 8 to 22 carbon atoms, such as, for example, a 2-ethylhexanoyl, caproyl, lauroyl, myristoyl, palmitoyl, stearoyl or cocoyl group. The salts of these compounds can be aluminum, magnesium, calcium, zirconium, zinc, sodium or potassium salts. The amino acid can, for example, be lysine, glutamic acid or alanine.

The term “alkyl” cited in the abovementioned compounds denotes in particular an alkyl group having from 1 to 30 carbon atoms and preferably having from 5 to 16 carbon atoms.

Hydrophobic treated pigments are described in particular in Application EP-A-1 086 683.

### Pearlescent Agents.

As used herein, the term “pearlescent agent” refers to colored particles of any shape, which are or are not iridescent, and which may be of natural or synthetic origin, and which exhibit a color effect via optical interference.

Mention may be made, as examples of pearlescent agents, of pearlescent pigments, such as titanium oxide-coated mica covered with an iron oxide, mica covered with bismuth oxychloride, titanium oxide-coated mica covered with chromium oxide, titanium oxide-coated mica covered with an organic dye, in particular of the abovementioned type, and also pearlescent pigments based on bismuth oxychloride.

They can also be mica particles, at the surface of which are superimposed at least two successive layers of metal oxides and/or of organic colorants.

The pearlescent agents can more particularly have a yellow, pink, red, bronze, orangey, brown, gold and/or coppery color or glint.

Mention may be made, by way of illustration of the pearlescent agents which can be introduced as interference pigment into the first composition, of gold-colored pearlescent agents sold in particular by BASF under the name Brilliant Gold 212G (TIMICA® pigment), Gold 222C (CLOISONNÉ® pigment), Sparkle Gold (TIMICA® pigment) and Monarch Gold 233X (CLOISONNÉ® pigment); bronze pearlescent agents sold in particular by Merck under the names Bronze Fine (17384) (COLORONA® pigment) and Bronze (17353) (COLORONA® pigment) and by BASF under the name Super Bronze (CLOISONNÉ® pigment); orange pearlescent agents sold in particular by BASF under the name Orange 363C (CLOISONNÉ® pigment) and by Merck under the names Passion Orange (COLORONA® pigment) and Matte Orange (17449) (MICRONA® pigment); brown-colored pearlescent agents sold in particular by BASF under the names Nu-Antique Copper 340XB (CLOISONNE® pigment) and Brown CL4509 (CHROMA-LITE® pigment); pearlescent agents with a copper glint sold in particular by BASF under the name Copper 340A (TIM ICA® pigment); pearlescent agents with a red glint sold in particular by Merck under the name Sienna Fine (17386) (COLORONA® pigment); pearlescent agents with a yellow glint sold in particular by BASF under the name Yellow (4502) (CHROMA-LITE® pigment); red-colored pearlescent agents with a gold glint sold in particular by BASF under the name Sunstone G012 (GEMTONE® pigment), pink pearlescent agents sold in particular by BASF under the name Tan Opal G005 (GEMTONE® pigment); black pearlescent agents with a gold glint sold in particular by BASF under the name Nu-Antique Bronze 240 AB (TIMICA® pigment); blue pearlescent agents sold in particular by Merck under the name Matte Blue (17433) (MICRONA® pigment); white pearlescent agents with a silvery glint sold in particular by Merck under the name XIRONA® Silver pigment, and golden green pinkish orangey pearlescent agents sold in particular by Merck under the name Indian Summer (XIRONA® pigment); and their mixtures.

### Goniochromatic Pigments.

As used herein, the term “goniochromatic pigment” refers to a pigment which makes it possible to obtain, when the composition is spread over a substrate, a color distance in the a*b* plane of the CIE 1976 colorimetric space which corresponds to a variation Dh° in the angle of hue h° of at least 20° when the angle of observation is varied with respect to the normal by between 0° and 80°, for an angle of incidence of the light of 45°.

The color distance can be measured, for example, using a spectrogonioreflectometer of the Instrument Systems brand and with the GON 360 Goniometer reference, after the composition has been spread in the fluid state with a thickness of 300 μm using an automatic spreader over a contrast chart of the Erichsen brand and with the Type 24/5 reference, the measurement being carried out on the black background of the chart.

The goniochromatic pigment can be chosen, for example, from multilayer interference structures and liquid crystal coloring agents.

In the case of a multilayer structure, the latter can comprise, for example, at least two layers, each layer being produced, for example, from at least one material chosen from the group consisting of the following materials: MgF2, CeF3, ZnS, ZnSe, Si, SiO₂, Ge, Te, Fe₂O₃, Pt, Va, Al2O3, MgO, Y2O3, S2O3, SiO, HfO2, ZrO2, CeO2, Nb2O5, Ta2O5, TiO2, Ag, Al, Au, Cu, Rb, Ti, Ta, W, Zn, MoS2, cryolite, alloys, polymers and their combinations.

The multilayer structure may or may not exhibit, with respect to a central layer, a symmetry with regard to the chemical nature of the stacked layers.

Different effects are obtained according to the thickness and the nature of the various layers.

Examples of symmetrical multilayer interference structures are, for example, the following structures: Fe2O3/SiO2/Fe2O3/SiO2/Fe2O3, a pigment having this structure being sold under the name SICOPEARL® pigments by BASF; MoS2/SiO2/mica-oxide/SiO2/MoS2; Fe2O3/SiO2/mica-oxide/SiO2/Fe2O3; TiO2/SiO2/TiO2 and TiO2/Al2O3/TiO2, pigments having these structures being sold under the name XIRONA® pigments by Merck.

The liquid crystal coloring agents comprise, for example, silicones or cellulose ethers to which mesomorphic groups are grafted. Use may be made, as liquid crystal goniochromatic particles, for example, of those sold by Chenix and of those sold under the name HELICONE® HC effect pigments by Wacker.

Use may also be made, as goniochromatic pigment, of certain pearlescent agents, effect pigments on a synthetic substrate, in particular a substrate of alumina, silica, borosilicate, iron oxide or aluminum type, or interference glitter resulting from a polyterephthalate film.

Mention may in particular be made, as non-limiting examples of goniochromatic pigments, alone or as mixtures, of the goniochromatic pigments SunShine® sold by Sun Chemical, Cosmicolor Celeste® from Toyo Aluminum K.K., Xirona® from Merck and Reflecks Multidimensions® from BASF.

### Dyes.

The composition according to the invention may also comprise one or more dyes.

Among the liposoluble dyes, mention may be made especially of fluoran dyes such as, for example, red Sudan, FDC Red 4, DC Red 17, Red 21, Red 27, DC Green 6, Sudan brown, Yellow 10, DC Yellow 11, DC Violet 2, DC Orange 4, DC Orange 5, Yellow quinoline, or mixtures thereof.

### Preservative.

The composition may include a preservative. The preservative may include an organic acid preservative. Non-limiting examples of preservatives include benzoic acid and alkali metal and ammonium salts thereof (e.g. sodium benzoate), sorbic acid and alkali metal and ammonium salts thereof (e.g. potassium sorbate}, p-Anisic acid and alkali metal, and ammonium salts thereof, and salicylic acid and alkali metal and ammonium salts thereof, phenoxyethanol, benzyl alcohol, salicylic acid, sodium benzoate, caprylyl glycol, methyl paraben, propyl paraben, ethylhexylglycerin, 1,2-propanediol and any mixture thereof.

The preservative may be present in a total amount of no more than 2% by weight of the composition. The preservative may be present in a total amount of no more than 1.5% by weight of the composition. The preservative may be present in a total amount of no more than 1% by weight of the composition. The composition may be substantially free of preservatives.

### Skin Care Agent.

The composition may include a skin care agent. The term “skin care agent” means an agent for enhancing the aesthetic appearance and/or comfort of the skin.

The skin care agent may be ceramide. Ceramides are a family of waxy lipid molecules that are composed of sphingosine and a fatty acid. Ceramides include ceramide 1, ceramide 2, ceramide 3, ceramide 4, ceramide 5, ceramide IA, ceramide 6 II, ceramide AP, ceramide EOP, ceramide EOS, ceramide NP, ceramide NG, ceramide NS, ceramide AS, and ceramide NS dilaurate.

The composition may include a ceramide in a total amount of no more than 2% by weight of the composition. The composition may include a ceramide in a total amount of no more than 1.5% by weight of the composition. The composition may include a ceramide in a total amount of no more than 1% by weight of the composition. The composition may include a ceramide in a total amount of no more than 0.5% by weight of the composition. The composition may include a ceramide in a total amount of no more than 0.25% by weight of the composition.

The skin care agent may include a probiotic lysate. A probiotic is known, traditionally, as “microorganisms that, when administered in adequate amounts, confer a health benefit on the host.” Here, the composition use lysates of such microorganisms. A non-limiting example of such a lysate is bifida ferment lysate.

The probiotic lysate may be present in a total amount of no more than 2% by weight of the composition. The probiotic lysate may be present in a total amount of no more than 1.5% by weight of the composition. The probiotic lysate may be present in a total amount of no more than 1% by weight of the composition. The composition may be substantially free of a probiotic lysate.

The skin care agent may include hyaluronic acid or a derivative thereof. In some embodiments, only a single hyaluronic acid or a derivative thereof is present. In some embodiments, a plurality of hyaluronic acid or a derivative thereof are present.

In the context of the present invention, the term “hyaluronic acid or a derivative thereof” covers the basic unit of hyaluronic acid which includes the smallest fraction of hyaluronic acid comprising a disaccharide dimer, namely D-glucuronic acid and N-acetylglucosamine.

The term “hyaluronic acid or a derivative thereof” also comprises, in the context of the present invention, the linear polymer comprising the polymeric unit described above, linked together in the chain via alternating beta(1,4) and beta(1,3) glycosidic linkages, having a molecular weight (MW) that can range between 380 and 13,000,000 daltons (Da). This molecular weight depends in large part on the source from which the hyaluronic acid is obtained and/or on the preparation methods.

The term “hyaluronic acid or a derivative thereof” also comprises, in the context of the present invention, the hyaluronic acid salts, and in particular the alkali metals salts such as the sodium salt and the potassium salt.

In the natural state, hyaluronic acid is present in pericellular gels, in the base substance of the connective tissues of vertebrate organs such as the dermis and epithelial tissues, and in particular in the epidermis, in the synovial fluid of the joints, in the vitreous humor, in the human umbilical cord and in the Crista galli apophysis.

Thus, the term “hyaluronic acid or a derivative thereof” comprises all the fractions or subunits of hyaluronic acid having a molecular weight in particular within the molecular weight range recalled above.

According to a preferred embodiment of the invention the hyaluronic acid fractions suitable for the use covered by the present invention have a molecular weight of between 50,000 and 5,000,000, in particular between 100,000 and 5,000,000, especially between 400,000 and 5,000,000 Da. In this case, the term used is high-molecular-weight hyaluronic acid.

Alternatively, the hyaluronic acid fractions that may also be suitable for the use in the present invention are chosen from those with a molecular weight of between 50,000 and 400,000 Da (intermediate-molecular-weight hyaluronic acid) and from those with a molecular weight of less than 50,000 Da (low-molecular-weight hyaluronic acid).

Finally, the term “hyaluronic acid or a derivative thereof” also comprises hyaluronic acid esters in particular those in which all or some of the carboxylic groups of the acid functions are esterified with oxyethylated alkyls or alcohols, containing from 1 to 20 carbon atoms, in particular with a degree of substitution at the level of the D-glucuronic acid of the hyaluronic acid ranging from 0.5 to 50 percent. Mention may in particular be made of methyl, ethyl, n-propyl, n-pentyl, benzyl and dodecyl esters of hyaluronic acid.

In some embodiments, the hyaluronic acid or a derivative thereof may be present in a total amount that is no more than about 2% by weight of the composition. In some embodiments, the hyaluronic acid or a derivative thereof may be present in a total amount that is no more than about 1% by weight of the composition. In some embodiments, the hyaluronic acid or a derivative thereof may be present in a total amount that is no more than about 0.5% by weight of the composition. In some embodiments, the hyaluronic acid or a derivative thereof may be present in a total amount that is no more than about 0.25% by weight of the composition. In some embodiments, the hyaluronic acid or a derivative thereof may be present in a total amount that is no more than about 0.1% by weight of the composition. In some embodiments, the composition may be substantially free of hyaluronic acid or a derivative thereof.

The skin care agent may include at least one hydrophilic active agent chosen from C-glycosides or derivatives thereof of the following general formula (I):

S—CH2-X—R (I)
in which: R denotes an unsubstituted linear C₁-C₄ and especially C₁-C₂ alkyl radical, in particular methyl; S represents a monosaccharide chosen from D-glucose, D-xylose, N-acetyl-D-glucosamine and L-fucose, and in particular D-xylose; X represents a group chosen from —CO—, —CH(OH)— and —CH(NH₂)— and preferentially a —CH(OH)— group; and also the cosmetically acceptable salts thereof, solvates thereof such as hydrates, and optical isomers thereof.

As non-limiting illustrations of C-glycoside derivatives of formula (I) that are more particularly suitable for the invention, mention may especially be made of the following compounds: C-beta-D-xylopyranoside-n-propan-2-one; C-alpha-D-xylopyranoside-n-propan-2-one; C-beta-D-xylopyranoside-2-hydroxypropane; C-alpha-D-xylopyranoside-2-hydroxypropane; 1-(C-beta-D-glucopyranosyl)-2-hydroxypropane; 1-(C-alpha-D-glucopyranosyl)-2-hydroxypropane; 1-(C-beta-D-glucopyranosyl)-2-aminopropane; 1-(C-alpha-D-glucopyranosyl)-2-aminopropane; 3′-(acetamido-C-beta-D-glucopyranosyl)propan-2′-one; 3′-(acetamido-C-alpha-D-glucopyranosyl)propan-2′-one; 1-(acetamido-C-beta-D-glucopyranosyl)-2-hydroxypropane; 1-(acetamido-C-beta-D-glucopyranosyl)-2-aminopropane; and also the cosmetically acceptable salts thereof, solvates thereof such as hydrates, and optical isomers thereof.

According to a particular embodiment, a C-glycoside of formula (I) that is suitable for the invention may advantageously be C-beta-D-xylopyranoside-2-hydroxypropane, the INCI name of which is Hydroxypropyl Tetrahydropyrantriol, sometimes referred to as proxylane.

The salts of the C-glycosides of formula (I) that are suitable for the invention may comprise conventional physiologically acceptable salts of these compounds, such as those formed from organic or inorganic acids. Examples that may be mentioned include the salts of mineral acids, such as sulfuric acid, hydrochloric acid, hydrobromic acid, hydriodic acid, phosphoric acid and boric acid. Mention may also be made of the salts of organic acids, which may comprise one or more carboxylic, sulfonic or phosphonic acid groups. They may be linear, branched or cyclic aliphatic acids, or alternatively aromatic acids. These acids may also comprise one or more heteroatoms chosen from O and N, for example in the form of hydroxyl groups. Mention may especially be made of propionic acid, acetic acid, terephthalic acid, citric acid and tartaric acid.

The solvates that are acceptable for the compounds described above comprise conventional solvates such as those formed during the final step of preparation of said compounds due to the presence of solvents. Examples that may be mentioned include solvates due to the presence of water or of linear or branched alcohols, such as ethanol or isopropanol.

According to one embodiment, the composition according to the invention comprises a C-glycoside in an amount of between 0.01% and 10% by weight of active material (C-glycoside) relative to the total weight of the composition. In some embodiments, the C-glycoside may be present in a total amount of less than 5% by weight. In some embodiments, the C-glycoside may be present in a total amount of less than 4% by weight. In some embodiments, the C-glycoside may be present in a total amount of less than 3% by weight. In some embodiments, the C-glycoside may be present in a total amount of less than 2% by weight. In some embodiments, the composition may be substantially free of the C-glycoside. In some embodiments, the composition may be free of the C-glycoside.

The skin care agent may include a peptide, such as a pentapeptide derivative (e.g., MATRIXIL® palmitoyl pentapeptide, synthetic peptides such as iamin, biopeptide CL, or a palmitoyloligopeptide, or peptides extracted from plants, such as the soybean hydrolysate, rice peptide, or peptide extracted from hazelnut.

According to one embodiment, the composition according to the invention comprises a peptide in an amount of between 0.01% and 10% by weight of a peptide relative to the total weight of the composition. In some embodiments, the peptide may be present in a total amount of less than 5% by weight. In some embodiments, the peptide may be present in a total amount of less than 4% by weight. In some embodiments, the peptide may be present in a total amount of less than 3% by weight. In some embodiments, the peptide may be present in a total amount of less than 2% by weight. In some embodiments, the composition may be substantially free of the peptide. In some embodiments, the composition may be free of the peptide.

### Antioxidant.

The composition may include an antioxidant. In some embodiments, the composition may include lipophilic antioxidant(s). The lipophilic antioxidants may be tocopherol, pentaerythrityl tetra-di-t-butyl hydroxyhydrocinnamate, or a mixture thereof.

The antioxidant may be present in a total amount of no more than 2% by weight of the composition. The antioxidant may be present in a total amount of no more than 1.5% by weight of the composition. The antioxidant may be present in a total amount of no more than 1% by weight of the composition. The composition may be substantially free of antioxidants.

In various embodiments, a method may be provided. The method may include providing a cosmetic composition as disclosed herein. The method may include applying the cosmetic composition to a lip or skin.

### Other Materials.

The composition may include one or more additional materials. The one or more additional materials may include any other cosmetically acceptable material. In some embodiments, the composition may include no more than 10% of the one or more additional materials. In some embodiments, the composition may include no more than 9% of the one or more additional materials. In some embodiments, the composition may include no more than 8% of the one or more additional materials. In some embodiments, the composition may include no more than 7% of the one or more additional materials. In some embodiments, the composition may include no more than 6% of the one or more additional materials. In some embodiments, the composition may include no more than 5% of the one or more additional materials. In some embodiments, the composition may include no more than 4% of the one or more additional materials. In some embodiments, the composition may include no more than 3% of the one or more additional materials. In some embodiments, the composition may include no more than 2% of the one or more additional materials. In some embodiments, the composition may be free, or substantially free, of the one or more additional materials.

### Filler.

The one or more additional materials may include a filler. The filler may be present in a total amount of not more than 5% by weight of the composition. The filler may be present in a total amount of not more than 4% by weight of the composition. The filler may be present in a total amount of not more than 3% by weight of the composition. The filler may be present in a total amount of not more than 2% by weight of the composition. The filler may be present in a total amount of not more than 1% by weight of the composition.

The filler may be a mineral filler, an organic filler, or an optionally modified clay.

Mineral Filler and/or Organic Filler

In some embodiments, the filler may be colorless or white solid particles of any form, which are in an insoluble form dispersed in the medium of the composition. These particles, of mineral or organic nature, give body or rigidity to the composition and/or softness and uniformity to the makeup.

The fillers used in the compositions according to the invention may have a lamellar, globular, spherical, platelet, or fibrous form or of any other form intermediate between these defined forms.

The fillers according to the invention may or may not be surface-coated, and in particular they may be surface-treated with silicones, amino acids, fluorinated derivatives or any other substance which promotes the dispersion and the compatibility of the filler in the composition.

Examples of mineral fillers that may be mentioned include talc, mica, silica, hollow silica microspheres, kaolin, calcium carbonate, magnesium carbonate, hydroxyapatite, boron nitride, glass or ceramic microcapsules, or composites of silica and of titanium dioxide, for instance the TSG series sold by Nippon Sheet Glass

Examples of organic fillers that may be mentioned include polyethylene powders, polymethyl methacrylate powders, polytetrafluoroethylene powders, acrylic acid copolymer powders (Polytrap from the company Dow Corning), lauroyl lysine, hollow polymer microspheres such as those of polyvinylidene chloride/acrylonitrile, for instance Expancel (Nobel Industrie), hexamethylene diisocyanate/trimethylol hexyllactone copolymer powder (Plastic Powder from Toshiki), silicone resin microbeads (for example Tospearl from Toshiba), synthetic or natural micronized waxes, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate, Polypore® L 200 (Chemdal Corporation), polyurethane powders, in particular powders of crosslinked polyurethane comprising a copolymer, said copolymer comprising trimethylol hexyllactone.

The mineral and/or organic filler may be present in a total amount of not more than 5% by weight of the composition. In some embodiments, the composition may be substantially free of any mineral and/or organic filler

### Optionally Modified Clay.

The filler may be an optionally modified clay. Clays are silicates containing a cation which can be chosen from calcium, magnesium, aluminium, sodium, potassium or lithium cations, and their mixtures.

Mention may be made, as examples of such products, of clays of the family of the smectites, and also of the family of the vermiculites, stevensites or chlorites. These clays can be of natural or synthetic origin.

Preferably, use is made of organophilic clays, more particularly of modified clays, such as montmorillonite, bentonite, hectorite, attapulgite or sepiolite, and their mixtures. The clay is preferably a bentonite or a hectorite.

These clays are modified with a chemical compound chosen from quaternary amines, tertiary amines, amine acetates, imidazolines, amine soaps, fatty sulfates, alkylarylsulfonates or amine oxides, and their mixtures.

Mention may thus be made of hectorites modified by a quaternary amine, more specifically by a C₁₀ to C₂₂ fatty acid ammonium halide, such as a chloride, comprising or not comprising an aromatic group, such as hectorite modified by a distearyldimethylammonium halide, preferably a chloride, (CTFA name: Disteardimonium hectorite), such as, for example, those sold under the name Bentone 38V, Bentone 38V CG or Bentone EW CE by Elementis, or stearalkonium hectorites, such as in particular the product Bentone 27 V.

Mention may also be made of quaternium-18 bentonites, such as those sold, inter alia, under the names Bentone 34 by Elementis, Claytone 40, Tixogel VP by United Catalyst by Southern Clay; stearalkonium bentonites, such as those sold under the names Tixogel LG by United Catalyst and Claytone AF and Claytone APA by Southern Clay; or quaternium-18/benzalkonium bentonites, such as those sold under the name Claytone HT by Southern Clay.

The optionally modified clay may be present in a total amount of not more than 5% by weight of the composition. The optionally modified clay may be present in a total amount of not more than 4% by weight of the composition. The optionally modified clay may be present in a total amount of not more than 3% by weight of the composition. The optionally modified clay may be present in a total amount of not more than 2% by weight of the composition. The optionally modified clay may be present in a total amount of not more than 1% by weight of the composition.

### EXAMPLES

One exemplary formula and four comparative formulas (see Table 1, below), were produced by combining the necessary materials, heating to around 70-75° C., mixing until homogenous, and cooling to room temperature.

TABLE 1
(Formulas)

| | Ex. 1 | Comp. 1 | Comp. 2 | Comp. 3 | Comp. 4 |
|---|---|---|---|---|---|
| Material | (% w/w) | (% w/w) | (% w/w) | (% w/w) | (% w/w) |
| Allyl Stearate/VA | 0.5-6% | 0.5-6% | 0.5-6% | — | 0.5-6% |
| Copolymer | | | | | |
| C12-22 alkyl acrylate/ | 0.5-6% | — | — | 0.5-6% | — |
| hydroxyethylacrylate | | | | | |
| Copolymer | | | | | |
| Hydrogenated Styrene/ | — | 0.5-6% | — | — | — |
| Butadiene Copolymer | | | | | |
| Poly C10-C30 Alkyl | — | — | — | — | 0.5-6% |
| Acrylate | | | | | |
| Emulsifying Agent or | 2-5% | 2-5% | 2-5% | 2-5% | 2-5% |
| Stabilizer | | | | | |
| Polyol | 1-10% | 1-10% | 1-10% | 1-10% | 1-10% |
| Fatty Substance - | 2-35% | 2-35% | 2-35% | 2-35% | 2-35% |
| Polyolefins/Hydrocarbons | | | | | |
| Fatty | 15-65% | 15-65% | 15-65% | 15-65% | 15-65% |
| Substance - Emollients | | | | | |
| Skin Care Active | 1-10% | 1-10% | 1-10% | 1-10% | 1-10% |
| Polymeric Elastomer | 0.5-10% | 0.5-10% | 0.5-10% | 0.5-10% | 0.5-10% |
| Dispersing Agent | 1-6% | 1-6% | 1-6% | 1-6% | 1-6% |
| Hydrocarbon-based film | 3-9% | 3-9% | 3-9% | 3-9% | 3-9% |
| former | | | | | |
| Antioxidant | <1% | <1% | <1% | <1% | <1% |
| Colorants | 1-10% | 1-10% | <1% | <1% | <1% |
| | | | 1-10% | 1-10% | 1-10% |
| Polymer Wax | 1-3% | 1-3% | 1-3% | 1-3% | 1-3% |
| Preservative | <2% | <2% | <2% | <2% | <2% |
| Optionally Modified Clay | 1-2% | 1-2% | 1-2% | 1-2% | 1-2% |
| Other Excipients | <0.01% | <0.01% | <0.01% | <0.01% | <0.01% |

### Evaluation.

The above formulas were evaluated for shear viscosity, liquid-solid transition, using a rheometer and measured for stability under various temperature conditions.

Shear viscosity was measured on the sample from 0.01 to 1000/sec at 25° C. using a 2° degree cone and plate geometry using an TA Instrument Discovery Series Rheometer. For the liquid-solid transition, samples were placed under shear at 0.1/s at 25′° C. for 2 min, and then under high shear at 200/s for 10 minutes at 25′° C. before going to oscillation mode with 0.2%/strain and 1 rad/s frequency for 5 minutes. The liquid solid transformation is defined by the cross-over of the G′ and G″ behavior. For DSC, samples were equilibrated at 25° C., then heated to 70′° C. with a heating rate of 10 degrees/min. After maintaining at the target temperature for 2 minutes, the samples were then cooled down to −20° C. with a rate of −10 degrees/min, then heated again to 70′° C. at 10 degrees/min. For stability, samples were placed in sealed jars in temperature-controlled chambers, either in 50° C. for 8 weeks, or 45° C. for 4 weeks.

TABLE 2
(Evaluations)

| Material | Ex. 1 | Comp. 1 | Comp. 2 | Comp. 3 | Comp. 4 |
|---|---|---|---|---|---|
| Shear Viscosity | 2 order drop | 1 order drop | <1 order drop | 2 order drop | 2 order drop |
| (Pa/s) | with shear. | in shear | in shear | in sheer | in sheer |
| | | viscosity. | viscosity. | viscosity. | viscosity. |
| | | | Initial | Initial | Initial |
| | | | Viscosity < | Viscosity < | Viscosity > |
| | | | Ex. 1 | Ex. 1 | Ex. 1 |
| Liquid Solid | Crossover ~150 | Always more | Always more | Cross over | Solid from |
| Transformation | seconds. | liquid at rest | liquid at rest | from liquid | beginning |
| (1 radian/Hz) | | and shear. No | and shear. No | to solid | |
| | | crossover | crossover | around 75 | |
| | | point. | point. | seconds | |
| Differential scanning | 54° C. | No Data | No Data | No Data | ~40° C. |
| calorimetry (DSC) | | | | | |
| (Melting Point ° C.) | | | | | |
| Stability @ | Ok | Failed | Failed | Failed | Failed |
| 45° C. - 4 weeks | | | | | |

As seen, only the exemplary formula performed well across all evaluations. The exemplary formula exhibited good stability over time at elevated temperature, with low phase separation and low mobility on lips until it sets. All other formulations had poor stability testing results. Comparative formulations 1-3 had noticeable more mobility on lips until it sets. Comparative formulation 4 was simply solid from the beginning, and elevated temperatures were causing wax recrystallization, resulting in texture changes.

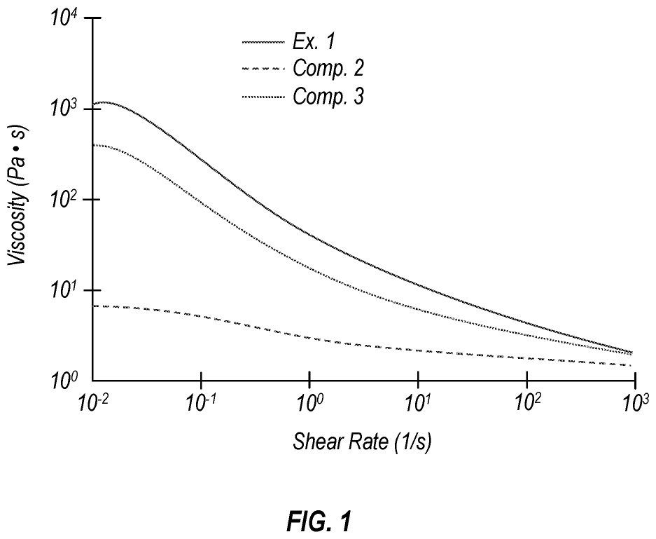

Looking at FIG. 1, it can be seen that, at low shear rates, there are synergies between the two copolymers. That is, the sum of the viscosity impact provided by the allyl stearate/VA copolymer alone (see Comp. 2) and the viscosity impact provided by the C12-22 alkyl acrylate/hydroxyethylacrylate copolymer alone (see Comp. 3) is less than the viscosity impact seen by the combination of the two copolymers (see Ex. 1).

Those skilled in the art will recognize or be able to ascertain using no more than routine experimentation many equivalents to the specific embodiments of the invention described herein. Such equivalents are intended to be encompassed by the following claims.

What is claimed is:

1. A cosmetic composition, comprising:
a C10-C40 alkyl acrylate polymer;
an allylic polymer;
a polar solvent; and
an emulsifying agent or stabilizer;
wherein the cosmetic composition is an emulsion.

2. The cosmetic composition of claim 1, wherein the C10-C40 alkyl acrylate polymer is a C12-22 alkyl acrylate/hydroxyethylacrylate copolymer.

3. The cosmetic composition of claim 2, wherein the allylic polymer is an allyl stearate/vinyl acrylate (VA) copolymer.

4. The cosmetic composition of claim 3, wherein the emulsifying agent or stabilizer is Polyglyceryl-4 Diisostearate/Polyhydroxystearate/Sebacate.

5. The cosmetic composition of claim 4, wherein the polar solvent is a polyol.

6. The cosmetic composition of claim 5, wherein the polyol is glycerin.

7. The cosmetic composition of claim 5, wherein the polyol comprises glycerin and a glyceryl ether and/or glyceryl ester.

8. The cosmetic composition of claim 5, further comprising a fatty substance, the fatty substance being present in a total amount of at least 30% by weight of the cosmetic composition.

9. The cosmetic composition of claim 8, wherein the fatty substance comprises diisostearoyl polyglyceryl-3 dimer dilinoleate.

10. The cosmetic composition of claim 8, further comprising a polymeric elastomer.

11. The cosmetic composition of claim 10, further comprising a dispersing agent.

12. The cosmetic composition of claim 11, further comprising a hydrocarbon-based resin.

13. The cosmetic composition of claim 12, further comprising a polyolefin, a colorant, a preservative, a skin care agent, and/or an antioxidant.

14. The cosmetic composition of claim 13, wherein the cosmetic composition is substantially free of water.

15. The cosmetic composition of claim 14, wherein the composition contains less than 5% by weight of the cosmetic composition all other materials.

16. A method, comprising:
providing a cosmetic composition according to claim 1; and
applying the cosmetic composition to a lip or skin.
